(19) United States
(12) Patent Application Publication
(10) Pub. No.: US 2025/0213672 A1
(43) Pub. Date: Jul. 3, 2025

(54) LIVE ATTENUATED JUNIN VIRUS AND RELATED COMPOSITIONS AND METHODS

(71) Applicants: Brian GOWEN, Logan, UT (US); Jack NUNBERG, Missoula, MT (US)

(72) Inventors: Brian GOWEN, Logan, UT (US); Jack NUNBERG, Missoula, MT (US)

(73) Assignee: UTAH STATE UNIVERSITY, Logan, UT (US)

(21) Appl. No.: 19/005,250

(22) Filed: Dec. 30, 2024

Related U.S. Application Data
(60) Provisional application No. 63/616,221, filed on Dec. 29, 2023.

(51) Int. Cl. A61K 39/12; A61K 39/00; C12N 7/00

(52) U.S. Cl. CPC A61K 39/12; C12N 7/00; A61K 2039/5254; C12N 2760/10021; C12N 2760/10022; C12N 2760/10034; C12N 2760/10062

(57) ABSTRACT

The present disclosure relates to a novel live-attenuated Junín virus (JUNV), which exhibits reduced likelihood for virulence reversion compared to the Candid#1 vaccine strain. The live-attenuated JUNV comprises at least three mutations in its GPC protein compared to a pathogenic strain of JUNV. One mutation is a substitution mutation at residue 427 like the Candid#1 vaccine strain. The live-attenuated JUNV comprises at least one mutation altering a NxT/S glycosylation motif in a region spanning residues 150-175 of the Junín virus GPC protein to inactivate the glycosylation site. In some embodiments, the live-attenuated JUNV further comprises a substitution mutation at residue 33. Compositions comprising the variant JUNV and methods of using the JUNV and compositions are also described herein.

### CROSS-REFERENCE TO RELATED APPLICATION

This application claims the benefit of U.S. Provisional Application No. 63/616,221, filed Dec. 29, 2023, the contents of which is hereby incorporated by reference.

### STATEMENT REGARDING FEDERALLY SPONSORED RESEARCH OR DEVELOPMENT

This invention was made with government support under R56 AI136467 and R01 AI150950 awarded by the National Institutes of Health.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 22,563-byte XML file named “007US-PAT” created on Dec. 20, 2024.

### TECHNICAL FIELD

The present disclosure relates to a live-attenuated variant of Junín virus with reduced risk for genetic reversion and residual pathogenicity that is suitable for use in vaccination, diagnostic, and therapeutic compositions.

### BACKGROUND

Arenaviruses (genus Mammarenavirus; family Arenaviridae) are endemic to rodent populations worldwide and some species can be transmitted to humans to cause severe life-threatening hemorrhagic fevers. These viruses can also spread person-to-person in healthcare settings and can be exported from endemic regions by infected travelers. In the absence of specific antiviral therapies and licensed vaccines, the hemorrhagic fever arenaviruses pose a significant threat to public health and national security. These threats are recognized in the classification of these viruses in the US as Category A priority pathogens.

Arenaviruses possess a bipartite single-strand RNA genome that is transcribed in an ambisense manner to generate four proteins: the GPC envelope glycoprotein and NP nucleoprotein are expressed from opposing reading frames on the S (small) genomic segment, whereas the Z matrix protein and L RNA-dependent RNA polymerase are similarly expressed from the L (large) segment. Arenavirus entry into the host cell is initiated by GPC binding to a cell-surface receptor, transferrin receptor 1 (TfR1) in the pathogenic New World arenavirus species and α-dystroglycan in the Old World species, followed by endocytic uptake of the virion particle. Upon acidification of the maturing endosome, GPC is triggered to drive fusion of the viral and endosomal membranes, depositing the ribonucleoprotein core of the virion in the cytosol to initiate infection. Unlike other class I viral fusion proteins, the mature GPC comprises three subunits: the conventional receptor-binding (GP1) and transmembrane fusion (GP2) subunits, as well as a unique 58-residue signal peptide (SSP). SSP is myristoylated at its cytosolic N terminus and contains two hydrophobic regions that span the membrane to form a hairpin structure. SSP interacts with GP2 to form a functional unit that senses endosomal pH and triggers the large-scale conformational change in GPC leading to membrane fusion. This interface is also targeted by multiple chemically distinct small-molecule fusion inhibitors. Interestingly, the attenuating F427I locus in the transmembrane domain (TMD) of Candid#1 GP2 interacts genetically with mutations in SSP to affect membrane fusion.

Of the pathogenic arenaviruses, the New World Junín virus (JUNV) is the only species for which a protective vaccine exists, albeit a vaccine used only in Argentina where the virus is endemic. The attenuated Candid#1 vaccine strain of JUNV was developed in the 1980s by a consortium of US and Argentine investigators to address the high incidence of fatal Argentine hemorrhagic fever (AHF) among agricultural workers. The vaccine virus was derived using a conventional attenuation strategy in which a pathogenic JUNV isolate (XJ) was serially passaged through guinea pigs, mice and in cell culture. The final Candid#1 strain was attenuated in mice, guinea pigs and rhesus macaques, and engendered protective immunity against lethal JUNV infection (S segment RNA reference genome: GenBank Accession No. AY746353.1, L RNA reference genome: GenBank Accession No. AY746354.1). Under the auspices of the U.S. Food and Drug Administration, the vaccine was proved to be safe and efficacious in human volunteers. The Candid#1 vaccine became available in Argentina in 1992 for seasonal use in at-risk agricultural workers and, as of 2006, more than a quarter million doses have been administered. The incidence of AHF in Argentina has dropped correspondingly. However, the vaccine is not licensed in the US, in part due to concerns regarding the genetic stability of the attenuation phenotype.

During the course of passage leading to Candid#1, the virus acquired a number of mutations in the envelope glycoprotein GPC (FIG. 1) and throughout the RNA genome. Recent reverse-genetics studies have localized a key determinant of attenuation to the phenylalanine-to-isoleucine substitution at position 427 (F427I) in the TMD of GP2. F427 is conserved in all pathogenic isolates of JUNV, and researchers have shown that virulence is abrogated upon introduction of the F427I mutation. The other amino-acid changes in Candid#1 GPC (see FIG. 1) did not significantly diminish virulence when introduced into the pathogenic isolate. The mechanism whereby I427 mediates attenuation is unknown.

Consistent with its attenuating phenotype, the F427I mutation in Candid#1 reduces virus fitness. Serial passage of recombinant Candid#1 (rCan) in cell culture readily gives rise to revertants at this position. Furthermore, intracranial rCan inoculation of neonatal mice, which is invariably fatal, results in the emergence of revertant viruses bearing the original F427. Although the Argentine Candid#1 vaccine has been reported to have a favorable safety profile, the number of doses administered is relatively low and follow up among immunized field workers has been incomplete. The reliance on a single nucleotide change at codon 427 for attenuation makes reversion all but inevitable.

Live attenuated viral vaccines provide strong and long-lived immunity but carry with them the risk that attenuating mutations may be prone to reversion to the more fit wild-type virus. This liability is best exemplified in the continuing effort to prevent reversion to virulence associated with the oral poliovirus (OPV) vaccine. Nonetheless, most of the best vaccines available are live attenuated viruses. In addition to the oral poliovirus vaccines, these include vaccines against the combination of mumps, measles, and rubella (MMR), smallpox, varicella, rotavirus, and yellow fever. Although reversion to a pathogenic form of Candid#1 has not been reported in the vaccination of over 250,000 at-risk Argentines, the number of persons receiving the vaccine and the extent of follow-up among agricultural workers is quite limited to conclude there is no risk of reversion. The relative dearth of Candid#1 immunizations stands in contrasts to the hundreds of millions of doses of OPV administered prior to recognition of virulent circulating vaccine-derived poliovirus revertants. Accordingly, attenuated variants of JUNV that can better resist genomic reversion are needed to meet the inevitable obstacle of the live attenuated Candid#1 virus vaccine losing its attenuated pathogenicity.

### SUMMARY OF THE DISCLOSURE

In one aspect, a variant of the Candid#1 virus vaccine strain is disclosed. The variant virus comprises at least one mutation in GP1 subunit of GPC protein in residue 166 (asparagine), residue 167 (proline), and/or residue 168 (alanine). In some aspects, the at least one mutation is selected from the group consisting of: N166Q, N166D, N166E, N166L, A168G, and A168V. The variant virus further comprises a mutation in SSP subunit of the GPC protein. In some aspects, the amino acid in residue 33 is not lysine. In some embodiments, the amino acid in residue 33 is a non-positively charged amino acid, for example, serine, alanine, glutamine, or glutamic acid. In some embodiments, the variant virus comprises the at least one mutation in the GP1 subunit and the SSP subunit of the GPC protein. In particular embodiments, the GPC protein has an amino acid sequence set forth in SEQ ID NO:1, SEQ ID NO:2, or SEQ ID NOs: 9-18.

In some aspects, an attenuated Junín virus (JUNV) is disclosed. The attenuated JUNV comprises at least one mutation that alters the NxT/S glycosylation motif in a region spanning residues 150-175 of the GPC protein thereby inactivating a glycosylation site, a mutation that targets an attenuating locus in the GPC protein's GP2 subunit, and a mutation that protects the mutation that targets an attenuating locus in the GPC protein's GP2 subunit from genetic reversion. Accordingly, in some aspects, an attenuated JUNV including at least three mutations in its GPC protein is disclosed. The first mutation alters a NxT/S glycosylation motif in a region spanning residues 150-175 of the GPC protein thereby inactivating a glycosylation site. The second mutation affects a different residue in the GPC protein's GP1 subunit than the first mutation or affects GPC protein's stable signal peptide subunit. The third mutation targets an attenuating locus in the GPC protein's GP2 subunit.

In some embodiments, the third mutation replaces a phenylalanine residue with an isoleucine.

In some embodiments, the first mutation replaces the T/S residue of the NxT/S glycosylation motif with a glycine or valine. In such embodiments, the second mutation alters the N residue of the NxT/S glycosylation motif to an amino acid selected from the group consisting of: glutamine, aspartic acid, and glutamic acid. Where the mutations are described in accordance with the amino acid sequence of clinical isolate XJ, the first mutation is T168G or T168V; the second mutation is N166Q, N166D, or N166E; and the third mutation is F427I. In certain embodiments, the attenuated JUNV further comprises a fourth mutation selected from the group consisting of: K33S, K33A, K33Q, and K33E. Alternatively, the second mutation affects GPC protein's stable signal peptide subunit, for example, replacing a lysin residue at position 33. In certain embodiments, the second mutation is selected from the group consisting of: K33S, K33A, K33Q, and K33E.

In some embodiments, the first mutation alters the N residue of the NxT/S glycosylation motif to an amino acid selected from the group consisting of: glutamine, aspartic acid, and glutamic acid. In such embodiments, the second mutation replaces a lysine residue in the stable signal peptide subunit. Where the mutations are described in accordance with the amino acid sequence of clinical isolate XJ, the first mutation is N166Q, N166D, or N166E; the second mutation is K33S, K33A, K33Q, and K33E; and the third mutation is F427I. Alternatively, the second mutation replaces the T/S residue of the NxT/S glycosylation motif with an alanine, glycine, or valine. Where the mutations are described in accordance with the amino acid sequence of clinical isolate XJ, the first mutation is N166Q, N166D, or N166E; the second mutation is T168A, T168G, or T168V; and the third mutation is F427I.

In some embodiments of the attenuated JUNV, the GPC protein has an amino acid sequence set forth in SEQ ID NO:1, SEQ ID NO:2, or SEQ NOs: 9-18.

In yet another aspect, a composition comprising the described variant virus or live-attenuated JUNV are disclosed. In some embodiments, the composition is a therapeutic composition or vaccine composition further comprising a pharmaceutically acceptable additive. In other embodiments, the composition is for diagnosing a subject has been infected with Junín virus, and the composition further comprises a surface, wherein the variant virus or live-attenuated JUNV, or proteins isolated thereof, is affixed to the surface.

In some aspects, a method of limiting reversion to virulence of Candid#1 vaccine strain is disclosed. The method comprises editing the genome of the Candid#1 vaccine strain resulting in inactivation of a NxT/S glycosylation motif in a region spanning residues 150-175 of the Junín virus GPC protein; editing the genome of the Candid#1 vaccine strain resulting in a mutation at a position corresponding to residue 33 of the Junín virus GPC protein; and expressing the edited genome of the Candid#1 vaccine strain in a mammalian cell thereby resulting in replication of an attenuated JUNV. The attenuated JUNV expresses a GPC protein with mutations at residues 33 and 427 and lacks NxT/S glycosylation motif in a region spanning residues 150-175.

In some aspects, the step of editing the genome of the JUNV resulting in inactivation of a NxT/S glycosylation motif introduces at least one amino acid substitution in the GPC protein selected from the group consisting of: N>Q, N>D, N>E, A>G, and A>V. In some aspects, the step of editing the genome editing the genome of the JUNV resulting in removal of a NxT/S glycosylation motif introduces at least one amino acid substitution in the GPC protein selected from the group consisting of: N166Q, N166D, N166E, A168G, and A168V. In some aspects, the step of editing the genome of the JUNV resulting in a mutation at a position corresponding to residue 33 replaces the lysine of residue 33 with serine, alanine, glutamine, or glutamic acid.

### BRIEF DESCRIPTION OF THE DRAWINGS

FIG. 1 is a schematic depicting the derivation of Candid#1. (Top) Passage history and selected intermediate isolates. GP=passages in guinea pigs; MB=passages in mouse brain; FRhL=passage in fetal rhesus lung epithelium cell culture. Virulence (v) or attenuation (a) in the respective species is shown below. (Bottom) Amino-acid substitutions in Candid#1 GPC. Positions 168 in GP1 and 427 in GP2 is highlighted, as these residues. This figure is adapted from Albariño et al., J Virol., 2011, 85(19):10404-10408 and reproduced from York and Nunberg, J Virol., 2017, 14;92(1):e01682-17.

FIG. 2 depicts, in accordance with certain embodiments, growth kinetics of rCan revertant viruses in cell culture. Vero cells were infected at a multiplicity of infection (MOI) of 0.01 with rCan, A168T rCan, I427F rCan, or A168T I427F rCan. Cell culture supernatants were collected every 24 h for 5 days and infectious virus concentrations were determined by enumeration of focus-forming units (FFU) in Vero cells.

FIGS. 3A and 3B depict, in accordance with certain embodiments, the pathogenesis of rCan revertant viruses in huTfR1 mice challenged with approximately 2000 CCID₅₀ of rCan, one of three rCan variants, or rRom. Mice (n=9−10/group) were administered intraperitoneally with approximately 2000 CCID₅₀ of rCan, one of three rCan variants, or rRom. Sham-infected animals (n=4) were included as normal controls. FIG. 3A is a graph showing the survival percentage during the course of the experiment. FIG. 3B is a graph showing the mean day of death and standard deviation of animals that succumbed to infection are shown. * indicates p <0.05, ** indicates p<0.01, *** indicates p<0.001, and **** indicates p<0.0001 compared to rRom-challenged mice by log-rank Mantel-Cox test for Kaplan-Meier plot and one-way ANOVA with Dunnett's posttest for the day of death analysis.

FIGS. 4A and 4B depict, in accordance with certain embodiments, the pathogenesis of rCan revertant viruses in huTfR1 mice challenged with approximately 20,000 CCID₅₀ of rCan, one of three rCan variants, or rRom. Mice (n=8−11/group) were administered intraperitoneally with 20,000 CCID₅₀ of rCan, one of three rCan variants, or rRom. Sham-infected animals (n=4) were included as normal controls. FIG. 4A is a graph showing the survival percentage during the course of the experiment. FIG. 4B is a graph showing the mean day of death and standard deviation of animals that succumbed to infection are shown. * indicates p<0.05, ** indicates p<0.01, and **** indicates p<0.0001 compared to rRom-challenged mice by log-rank Mantel-Cox test for Kaplan-Meier plot and one-way ANOVA with Dunnett's posttest for the day of death analysis.

FIGS. 5A-5C depict, in accordance with certain embodiments, the pathogenesis of rCan revertant viruses in guinea pigs challenged with approximately 2000 CCID₅₀ of rCan, one of three rCan variants, or rRom. Guinea pigs (n=6/virus infection group) were administered intraperitoneally with 2000 CCID₅₀ of rCan, A168T rCan, I427F rCan, A168T I427F rCan, or rRom. Survival (FIG. 5A), weight change (FIG. 5B), and body temperature (FIG. 5C) were monitored for 35 days. A sham-infected control animal was included for comparison. The body temperature data represent the group means and SEM. The percent changes in weights represent the group means and SEM for surviving animals relative to their starting weights on the day of virus challenge. ** indicates p=0.01 to 0.001 compared to guinea pigs infected with rRom, as determined using the Mantel-Cox log-rank test.

FIGS. 6A-6C depict, in accordance with certain embodiments, the pathogenesis of rCan revertant viruses in guinea pigs challenged with approximately 20,000 CCID₅₀ of rCan, one of three rCan variants, or rRom. Guinea pigs (n=6/virus infection group) were administered intraperitoneally with 20,000 CCID₅₀ of rCan, A168T rCan, I427F rCan, A168T I427F rCan, or rRom. Survival (FIG. 6A), weight change (FIG. 6B), and body temperature (FIG. 6C) were monitored for 35 days. A sham-infected control animal was included for comparison. The body temperature data represent the group means and SEM. The percent changes in weights represent the group means and SEM for surviving animals relative to their starting weights on the day of virus challenge. *** indicates p=0.001 to 0.0001 compared to guinea pigs infected with rRom, as determined using the Mantel-Cox log-rank test.

FIG. 7 depicts, in accordance with certain embodiments, the clinical scores of guinea pigs infected with 2000 CCID50 of rCan revertant viruses. Clinical score was recorded daily for each animal based on the presence or absence of weight loss exceeding 5% of the guinea pig's peak weight, 1° C. increase or decrease from the animal's baseline temperature, lethargy, ruffled fur, tremors, or paralysis. The clinical score represents the sum of the disease signs scored as 0 (not present) or 1 (present). Disease severity is depicted by increased intensity of red. Grey indicates the end of monitoring/scoring due to euthanasia/death.

FIG. 8 depicts focus formation by certain embodiments of the novel rCan viruses disclosed herein. The depicted rCan bear K33S mutation and a series of mutations at positions 166 and 168.

### DETAILED DESCRIPTION

Detailed aspects and applications of the disclosure are described below in the drawings and detailed description of the disclosure. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the disclosure. It will be understood, however, by those skilled in the relevant arts, that the present disclosure may be practiced without these specific details. It should be noted that there are many different and alternative configurations, devices, and technologies to which the disclosed disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

As used herein, the term “Candid#1 vaccine strain” refers to the live-attenuated Junín virus (JUNV) strain used to produce the JUNV vaccine used in Argentina. This strain is an attenuated JUNV derived from clinical isolate XJ is the inoculant. In some aspects, the amino acid sequence of the GPC protein of clinical isolate XJ is set forth in GenBank: AAB65462.1. In some aspects, the amino acid sequence of the GPC protein of the virus of the Candid#1 virus vaccine is set forth in GenBank: AAU34180.1.

As referenced herein, the term “viral genome from the virus of Candid#1 virus vaccine” refers to nucleic acid sequences with at least at least 95% sequence identity to GenBank Accession Nos. AY746353.1 or FJ969442.1, to GenBank Accession Nos. AY746354.1 or AY819707.2, or to GenBank Accession No. U70801.1. These sequences contain the nucleic acid sequence of the GPC protein in part or in whole.

As referenced herein, the term “revertant mutation” refers to an amino acid mutation compared to the sequence of Candid#1 that reverts the attenuation of the virus. In some aspects, revertant mutations include mutations in Candid#1 that restores the amino acid at a particular residue to the amino acid found in the wild-type thereby undoing attenuation of JUNV. For example, a mutation resulting in the attenuation of JUNV is F247I, so a revertant mutation can include I247F or any other substitution mutation on residue 247 where the change from isoleucine restores the virulence of the virus.

This disclosure is directed to an attenuated variant of Junín virus (JUNV) that is more able to resist genomic reversion that reverses attenuation. In some aspects, the disclosed attenuated variant of JUNV is a reversion-resistant variant of the Candid#1 vaccine strain comprising additional mutations that reduce the likelihood of the virus losing attenuation due to genomic changes as the virus replicates. As shown in Examples 1-3 herein, virulence of the Candid#1 vaccine strain is markedly increased if the mutations at positions 168 and 427 revert. Thus, by introducing a different mutation for position 168 or further mutation on position 166 and/or introducing a mutation that replaces lysine of residue 33, the likelihood of this attenuated variant losing attenuation is greatly reduced. The novel mutations disclosed for positions 166-168 better retains inactivation this NxT/S glycosylation site, and the mutation at K33 protects the F427I mutation from reversion. The disclosed variant also has low pathogenicity that makes it suitable for use in vaccination as a live-attenuated vaccine and in therapeutic compositions. The disclosed variant of JUNV is also suitable as a tool for diagnosing whether subject has been infected with JUNV due to its increased safety. Thus, in addition to the variant JUNV, vaccine, therapeutic, and diagnostic compositions comprising the variant JUNV are disclosed herein. In some aspects, the vaccine and therapeutic compositions further comprise a pharmaceutically acceptable carrier. In some aspects, the diagnostic composition comprises the disclosed attenuated JUNV and a surface, wherein the attenuated JUNV is affixed to the surface. In certain embodiments, the diagnostic composition is an immunoassay kit comprising the attenuated JUNV. In some embodiments, the diagnostic composition comprises a protein isolated from disclosed attenuated JUNV and a surface, wherein the protein isolate is affixed to the surface.

The attenuated variant of JUNV described herein comprises at least three mutations in its GPC protein, for example compared to clinical isolate XJ or any other wild-type pathogenetic JUNV. In some embodiments, the disclosed attenuated variant JUNV comprises a first mutation that alters a NxT/S glycosylation motif in a region spanning residues 150-175 of the GPC protein thereby inactivating a glycosylation site at the GP1 subunit; a second mutation at a different residue in the GPC protein's GP1 subunit than the first mutation that also alter the NxT/S glycosylation motif in residues 150-175; and a third mutation targeting an attenuating locus in the GPC protein's GP2 subunit. In other embodiments, the first mutation alters a NxT/S glycosylation motif in a region spanning residues 150-175 of the GPC protein thereby inactivating the glycosylation site; a second mutation at the GPC protein's stable signal peptide subunit; and a third mutation that targets an attenuating locus in the GPC protein's GP2 subunit.

In some implementations, the NxT/S glycosylation motif altered by the disclosed mutation is in a region spanning residues 160-170 of the GPC protein, spanning residues 165-170, spanning residues 166-169, or spanning residues 166-168. For example, in certain embodiments where the mutations are described in accordance with the amino acid sequence of clinical isolate XJ, the mutations altering the NxT/S glycosylation motif occurs at residue 166 (asparagine), residue 167 (proline), and/or residue 168 (threonine) to result in the inactivation of the glycosylation motif. In some aspects, N166 is replaced with an amino acid that will not support glycosylation (for example, D, Q, E, L) and/or T168 is replaced to inactivate the glycosylation site. In some aspects, the mutation inactivating the NxT/S glycosylation mutation is selected from at least one substitution mutation from the group consisting of: N>Q, N>D, N>E, N>L, A>G, A>V, T>A, T>G, and T>V. For the Candid#1 vaccine strain, the mutation T168A is already present to inactivate the glycosylation site. To ensure reversion to a functional NxT/S glycosylation motif is prevented in the Candid#1 vaccine strain, the reversion-resistant variant of the Candid#1 vaccine strain further comprises the aforementioned mutation at N166. In certain embodiments, mutation at residue 168 is changed to a substitution with glycine or valine, which requires more than one nucleotide change to revert the attenuating mutation (unlike the T168A mutation present on the Candid#1 vaccine strain). Accordingly, in particular embodiments, the mutation at residue 168 is T168G or T168V (or A168G or A168V based on the Candid#1 vaccine strain).

Accordingly in some embodiments, the first mutation and the second mutation are selected from a substitution mutation from the group consisting of: N166Q, N166D, N166E, N166L, A168G, A168V, T168A, T168G, T168V, S168A, S168G, and S168V. In particular embodiments, the mutations on the GP1 subunit of the GPC are selected from least one substitution mutation selected from the group consisting of: N166Q, N166D, N166E, A168G, A168V, T168G, and T168V.

The mutation at the GPC protein's stable signal peptide subunit replaces a lysine residue in the stable signal peptide subunit. In some aspects, the mutation replaces lysine of residue 33 with a non-positively charged amino acid (for example, serine, alanine, glutamine, or glutamic acid). In some implementations, the substitution mutation of K33 protects the attenuating mutation of F271I from reversion.

In some aspects, the mutation that targets an attenuating locus in the GPC protein's GP2 subunit replaces a phenylalanine residue with an isoleucine. Where the mutations are described in accordance with the amino acid sequence of clinical isolate XJ, the mutation that targets an attenuating locus in the GPC protein's GP2 subunit is F427I. This mutation is one of the attenuating mutations found in the Candid#1 vaccine strain.

Where the disclosed attenuated variant of JUNV is a reversion-resistant variant of the Candid#1 vaccine strain, the reversion-resistant variant comprises at least one mutation in GP1 subunit of GPC protein, wherein the at least one mutation is selected from the group consisting of: N166Q, N166D, N166E, N166L, A168G, and A168V and/or a mutation in residue 33 of the GPC protein, wherein the amino acid in residue 33 is not lysine and is a non-positively charged amino acid.

In certain embodiments of the attenuated variant of JUNV, the GPC protein has an amino acid sequence set forth in SEQ ID NO:1, SEQ ID NO:2, or SEQ ID NOs:9-18.

Also disclosed herein is a plasmid that encodes a mutated GPC protein, which can be used to produce a reversion-resistant variant of the Candid#1 vaccine strain using reverse genetics. Accordingly, the plasmid encodes the entire S-segment sequence of the Candid#1 vaccine strain with further mutations to the GPC protein. The plasmid comprises a nucleotide sequence that encodes a GPC protein from JUNV, wherein the nucleotide sequence comprises at least one nucleic acid mutation resulting in (1) removal of a NxT/S glycosylation motif in a region spanning residues 150-175 of the GPC protein of the virus of Candid#1 virus vaccine with mutations replace the N residue and the T/S residue; (2) replacement of lysine of residue 33 with a non-positively charged amino acid; or (3) inactivation of a NxT/S glycosylation motif in a region spanning residues 150-175 of the GPC protein of the virus of Candid#1 virus vaccine and replacement of lysine of residue 33 with a non-positively charged amino acid. The mutated GPC encoded by the plasmid has an isoleucine at residue 427. In some embodiments, the at least one nucleic acid mutation results in removal of the NxT/S glycosylation motif results in the at least change in the amino acid sequence of the GPC protein is selected from the group consisting of: N166Q, N166D, N166E, N166L, A168G, and A168V. In some embodiments, the at least one nucleic acid mutation replaces lysine of residue 33 with serine, alanine, glutamine, or glutamic acid. In some aspects, the nucleotide sequence has at least 95% sequence identity to GenBank Accession Nos. AY746353.1 or FJ969442.1, to GenBank Accession Nos. AY746354.1 or AY819707.2, or to GenBank Accession No. U70801.1. In certain embodiments, the plasmid comprises at least one nucleotide sequence selected from the group consisting of: SEQ ID NOs:3-8. In particular implementations, the plasmid comprises a viral genome encoding a GPC protein having the amino acid sequence set forth in SEQ ID NO:1, SEQ ID NO:2, or SEQ ID NOs:9-18. In some aspects, a composition for producing an attenuated JUNV is also described. The composition comprises the aforementioned plasmid and a second plasmid with a nucleotide sequence that encodes the entire JUNV L-segment sequence, a third plasmid with a nucleotide sequence that encodes JUNV NP protein, and a fourth plasmid with a nucleotide sequence that encodes JUNV L protein.

A method of limiting reversion to virulence of Candid#1 vaccine strain is also described. The method comprises editing the introducing mutations into the genome of Candid#1 vaccine strain resulting in (1) in a new mutation that inactivate the NxT/S glycosylation motif in a region spanning residues 150-175 of the Junín virus GPC protein and (2) a mutation at a position corresponding to residue 33 of the Junín virus GPC protein. The edited genome of the Candid#1 vaccine strain is then introduced into a mammalian cell for expression and replication of attenuated JUNV.

Expression and replication of the attenuated JUNV may be accomplished using reverse genetic systems well established in the art. Details of the reverse genetic systems may be found in Emonet et al., J Virol., 2011, 85(4):1473-83, the contents of which are incorporated by reference herein. Briefly, Reverse genetics to edit JUNV requires a plasmid expression the S-segment, a plasmid expressing the L-segment, and plasmids that just express N and L proteins. Expression of N and L proteins are needed to prime viral replication in the reverse-genetics protocol. As shown in the examples, L and S plasmids are first expressed using a plasmid promoter (PolI), and then the transcribed genomic L and S RNAs can replicate with N and L proteins.

In some implementations, the method comprises editing the genome of the Candid#1 vaccine strain resulting in at least one mutation that inactivate the NxT/S glycosylation motif in a region spanning residues 150-175 of the Junín virus GPC protein, wherein at least one mutation is selected from the group consisting of: N>Q, N>D, N>E, N>L, A>G, and A>V; editing the genome of the Candid#1 vaccine strain resulting in a mutation at a position corresponding to residue 33 of the Junín virus GPC protein; and expressing the edited genome of the Candid#1 vaccine strain in a mammalian cell thereby resulting in replication of an attenuated JUNV. The attenuated JUNV expresses a GPC protein with mutations at residues 33 and 427 and lacks NxT/S glycosylation motif in a region spanning residues 150-175.

In some aspects of the method, the removed NxT/S glycosylation motif is in a region spanning residues 160-170, 165-170, or 166-168 of the JUNV GPC protein. In certain implementations, the step of editing the genome editing the genome of the JUNV resulting in removal of a NxT/S glycosylation motif introduces at least one amino acid substitution in the GPC protein selected from the group consisting of: N166Q, N166D, N166E N166L, A168G, and A168V. In particular embodiments, the at least one amino acid substitution in the GPC protein is selected from the group consisting of: N166Q, N166D, N166E, A168G, and A168V.

In some aspects of the method, the step of editing the genome of the JUNV resulting in a mutation at a position corresponding to residue 33 replaces the lysine of residue 33 with serine, alanine, glutamine, or glutamic acid.

In a particular implementation, mammalian cells (such as BHK-21) are transfected with plasmids containing the complete L and S RNA segments of the bipartite Candid#1 genome in an antigenomic orientation, under transcriptional control of the mouse RNA polymerase I (Pol-I) promoter. The plasmid containing the complete S RNA segments of the bipartite Candid#1 genome comprises at least one nucleic acid sequences selected from the group consisting of SEQ ID NOs:3-8. Replication of the transcribed genomic RNAs is initiated through cotransfection with plasmids encoding the viral RNA-dependent RNA polymerase (L protein) and nucleoprotein (N protein). The resultant virus is amplified in permissive Vero cells. In some aspects, RT-PCR and Sanger sequencing is used to confirm retention of the expected GPC sequence, for example, a nucleic acid sequence encoding an amino acid sequence of any one of SEQ ID NO. 1, SEQ ID NO:2, and SEQ ID NOs:9-18.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1. Reversion at Position 427 in Candid#1 Does Not Restore Virulence

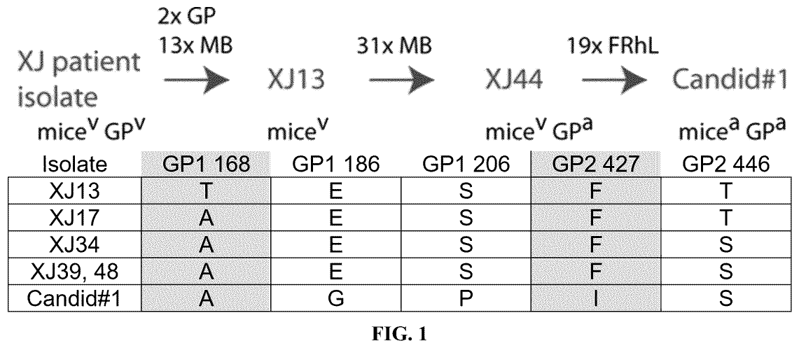

The F427I mutation in the transmembrane domain (TMD) of GP2 arose within a partially attenuated Candid#1 predecessor during the final series of attenuating passages in fetal rhesus lung (FRhL) cells (FIG. 1). This mutation, when introduced into a pathogenic strain of JUNV, attenuates the virus. Furthermore, this mutation readily reverts to the pathognomonic F427 upon serial passage of rCan in Vero cell culture, as well as upon intracranial inoculation in neonatal mice. These findings suggests that reversion may also occur in immunized persons and may result in the emergence of a pathogenic virus.

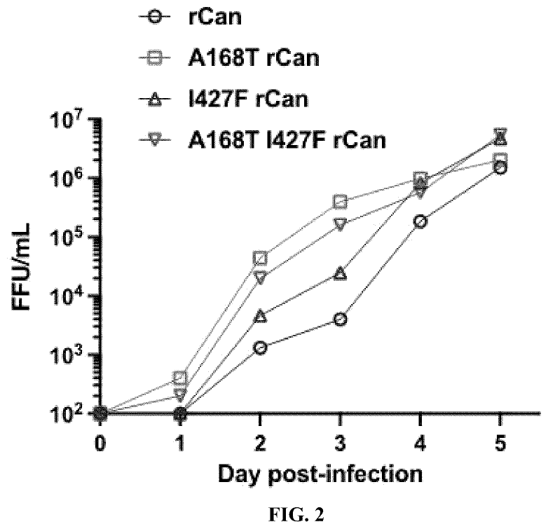

To assess whether reversion at this position is able to restore virulence to Candid#1, rCan bearing the I427F revertant mutation (I427F rCan) was constructed. This virus was readily rescued from plasmids expressing the anti-genomic S and L genomic RNA segments of Candid#1 using established reverse-genetics methodology. The sequence of the GPC open-reading-frame was confirmed using RT-PCR and Sanger sequencing, as previously described. As a precaution against possible virulence, these studies were performed under BSL3+ containment by Candid#1-immunized personnel. Consistent with the facile emergence of the I427F mutation upon passage in cell culture, I427F rCan grew more rapid than the parental rCan virus in Vero cells (FIG. 2). Final virus titers were similar between the viruses.

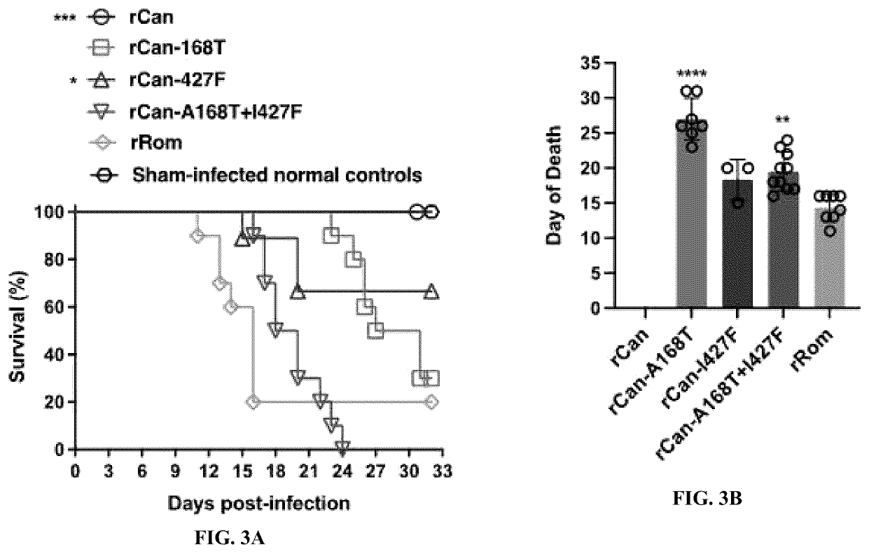

To assess the effect of the reversion on virulence, the recently developed huTfR1-knock-in mouse model of infection was employed. Expression of the human TfR1 orthologue renders the laboratory mouse susceptible to lethal infection by pathogenic Rom virus. By contrast, infection with 2000 CCID₅₀ (median cell culture infectious dose) of parental rCan is generally benign in this model (FIG. 3). The sole death in this group was unexpected and appeared unrelated to virus infection. Surprisingly, inoculation with the revertant I427F rCan resulted in only intermediate pathogenicity. Over the course of the study, 4 of 10 huTfR1 mice infected intraperitoneally with I427F rCan succumbed, versus 8 of 10 huTfR1 mice infected with recombinant Rom (rRom). Disease was temporally delayed in I427F rCan infection relative to rRom. All surviving animals showed similar weight gain trajectories regardless of the infecting virus; moribund animals shed significant weight prior to death or protocol-mandated euthanasia.

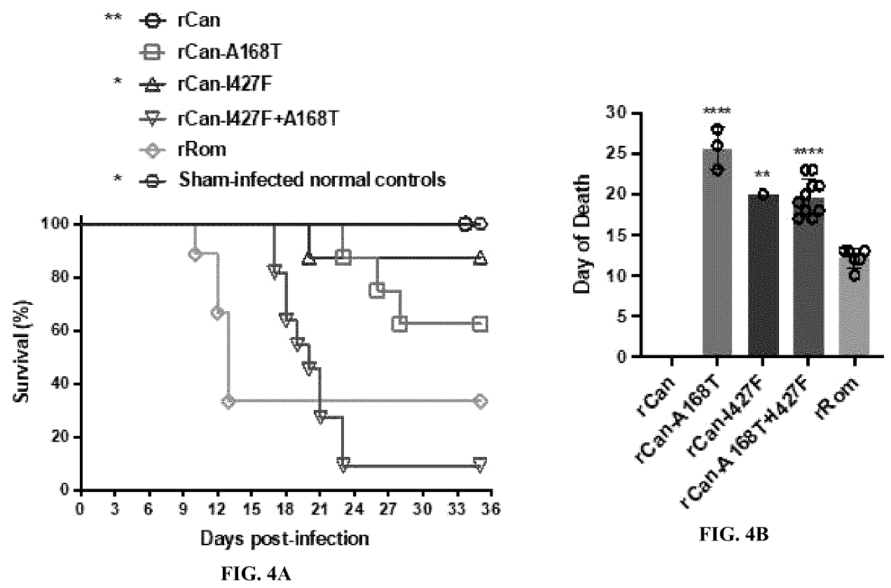

This study was repeated at a higher dose of virus (20,000 CCID₅₀) to investigate whether virulence is dependent on the inoculated dose (FIG. 4). Again, all rCan inoculated animals survived infection whereas rRom infection was fatal in 7 of 10 animals. And again, I427F rCan infection resulted in minimal lethality (1 of 10 huTfR1 mice). Clearly, the minimal virulence of the I427F rCan revertant is not related to the inoculating dose. The lesser mortality at the higher inoculating dose in this study likely reflects experimental variation.

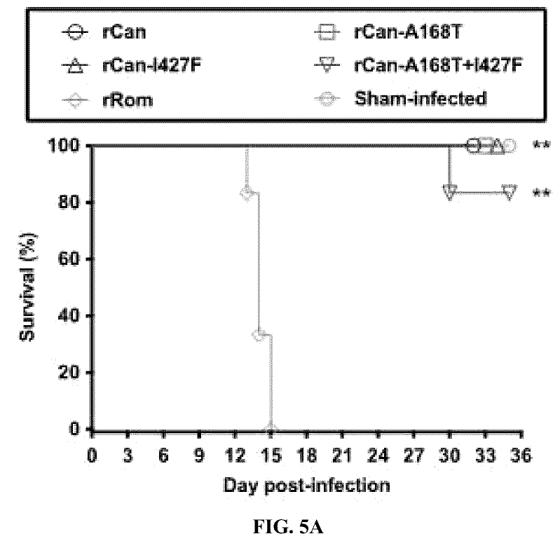

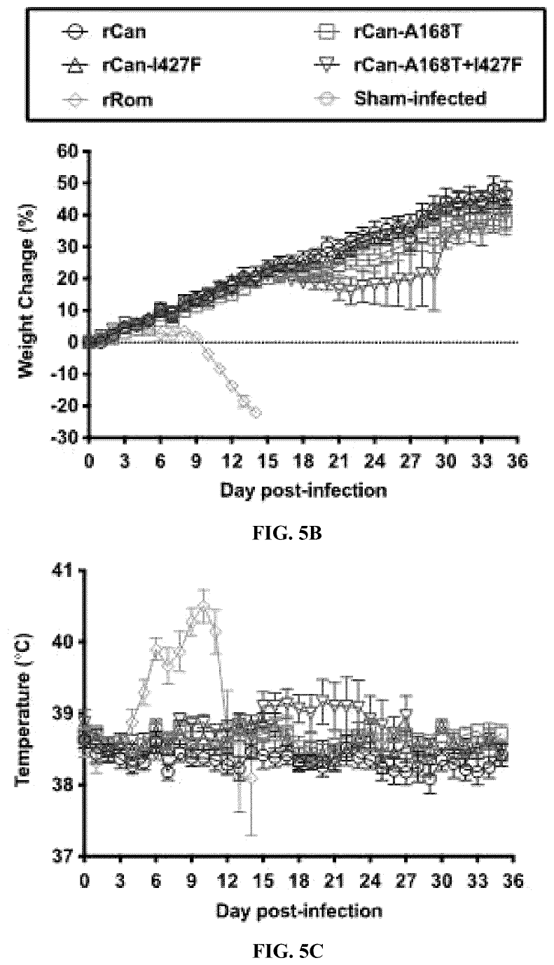

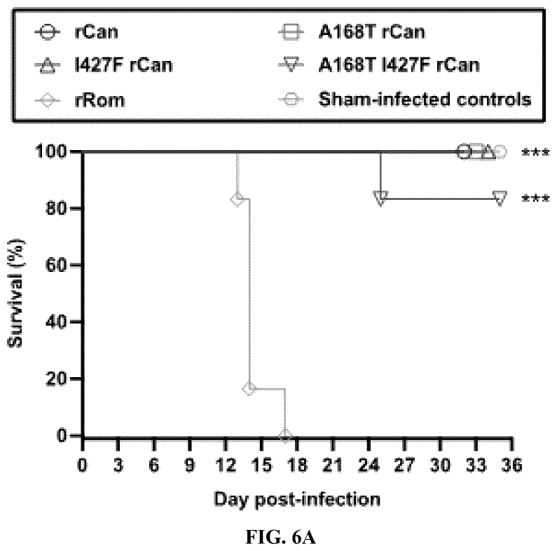

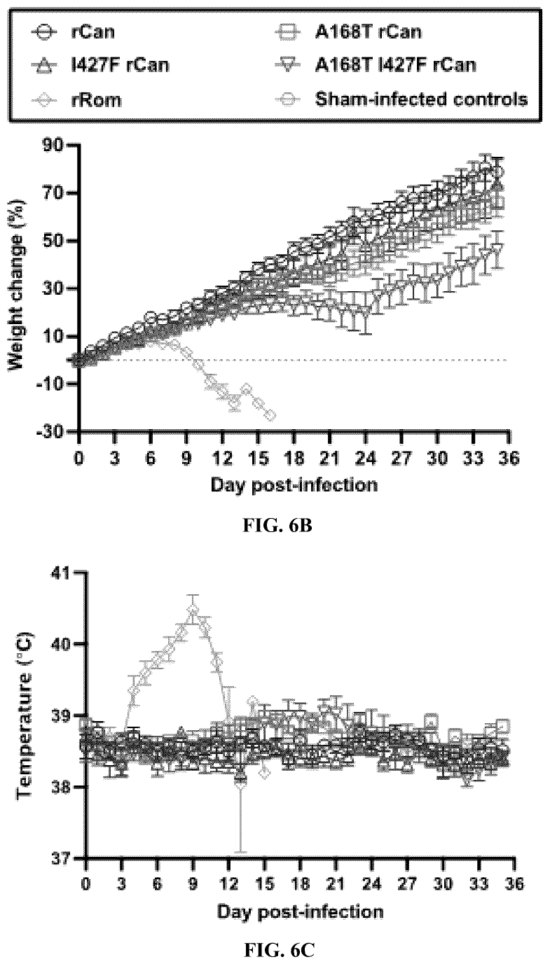

The Hartley guinea pig is a well-established model of JUNV infection and has been used to document attenuation in the derivation of Candid#1 and for quality-assurance in vaccine manufacture. To assess virulence of the I427F rCan revertant in this model, animals were inoculated intraperitoneally with 2,000 CCID₅₀ of virus. rRom infection resulted in 100% mortality in this model, whereas all rCan-inoculated animals survived, as did animals inoculated with the I427F rCan revertant (FIG. 5). This pattern was repeated upon inoculation with 20,000 CCID₅₀ of virus. None of the I427F rCan infected guinea pigs succumbed (FIG. 6). While the guinea pig appears to be less vulnerable to the I427F revertant virus than the huTfR1 mouse, both models demonstrate that reversion at this key determinant of Candid#1 attenuation does not result in a highly virulent virus.

### Example 2. Restoration of the N-Glycosylation Site at Position 166 in Candid#1 Does Not Restore Virulence

The T168A mutation in Candid#1 ablates a glycosylation site on GP1 thereby destabilizing GPC and resulting in reduced GPC accumulation and ER stress. Reversion at this position occurs readily upon passage of rCan in cell culture, and the double revertant virus (A168T and I427F) rapidly replaces the parental rCan in the virus population. Reversion at position 168 was also noted in brains of IFN-αβ/γ R−/− mice infected with a chimeric Machupo virus bearing the ectodomain of Candid#1 GPC (Koma et al., “Glycoprotein N-linked glycans play a critical role in arenavirus pathogenicity.” PLoS Pathog, 2021, 17:e1009356).

To investigate whether restoration of the glycosylation site at position 168 imparts virulence to Candid#1, the A168T revertant rCan was constructed. In keeping with its enhanced fitness, A168T rCan grew more rapidly than the parental rCan virus in Vero cell culture, much as I427F rCan (FIG. 2). In huTfR1 mice, A168T rCan was somewhat more virulent than I427F rCan (70% and 40% lethality at inoculating doses of 2,000 CCID₅₀ (FIG. 3) and CCID₅₀ FFU (FIG. 4), respectively). However, death due to A168T rCan infection was delayed by 5 days relative to the latter virus, and by 10 days relative to rRom. Despite significant virulence, the A168T rCan revertant virus did not match the extensive and rapid morbidity and mortality seen in rRom infection.

Further studies in guinea pigs again highlighted the difference in virulence between the revertant viruses and rRom. As with I427F rCan and in marked contrast to rRom, all guinea pigs inoculated with A168T rCan survived infection, at either inoculating dose (FIG. 5, 6). Taken together, neither A168T rCan or I427F rCan revertant viruses approached the early and extensive mortality seen in rRom infection.

### Example 3. Significant Virulence Requires Reversion at Both Attenuating Loci

The combination of the two revertant mutations might restore full virulence to the virus. The first indication of such an additive or synergistic effect was observed upon growth in Vero cell culture. A168T I427F rCan grew more rapidly than single revertant virus, albeit to similar end-point titers (FIG. 2).

In huTfR1 mice, inoculation with 2,000 CCID₅₀ or 20,000 CCID₅₀ of A168T I427F rCan resulted in 100% and 90% mortality, respectively, delayed somewhat relative to rRom but significantly earlier than the A168T revertant virus (FIG. 3, 4, respectively). The virulence of the double revertant virus in huTfR1 mice was significant and approaching that of the pathogenic rRom.

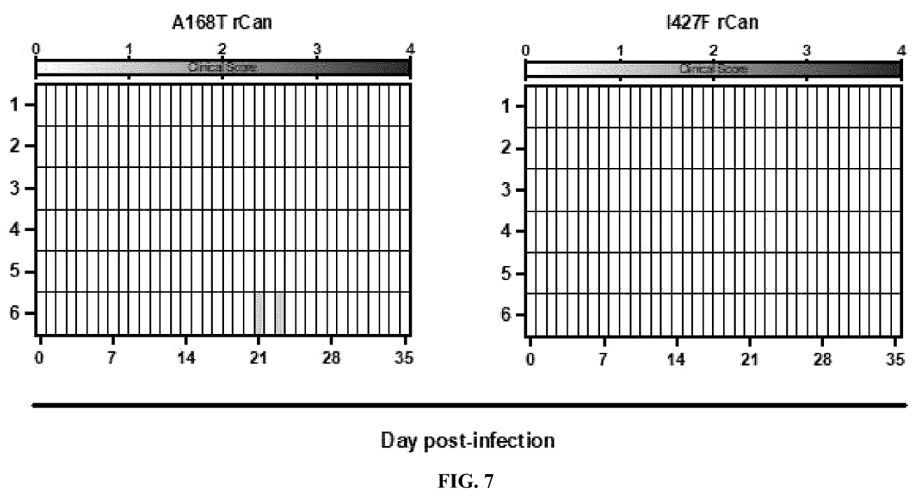

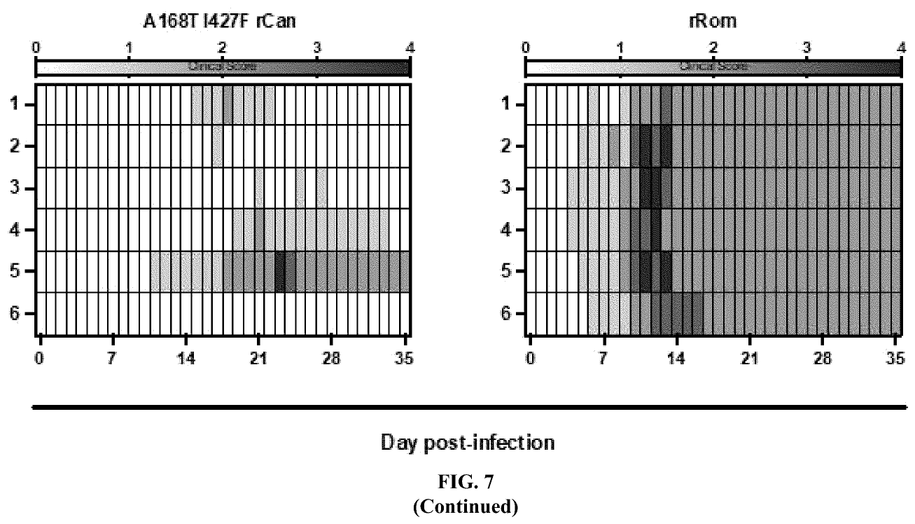

This enhanced virulence was also manifest in the guinea pig model. In contrast to either of the single revertant viruses, infection with the double revertant virus resulted in the death of 1 of 6 guinea pigs at either dose level (2,000 or 20,000 CCID₅₀) (FIG. 5, 6, respectively). rRom infection was uniformly lethal. Although mortality was limited, infection with the double revertant virus also resulted in significant morbidity in most animals, as judged by the duration and severity of clinical signs (FIG. 7). Based on these clinical scores, the A168T I427F rCan revertant to be roughly half as virulent as the highly pathogenic rRom control.

### Example 4. Viability of Certain Reversion-Resistant rCan Variants Disclosed Herein

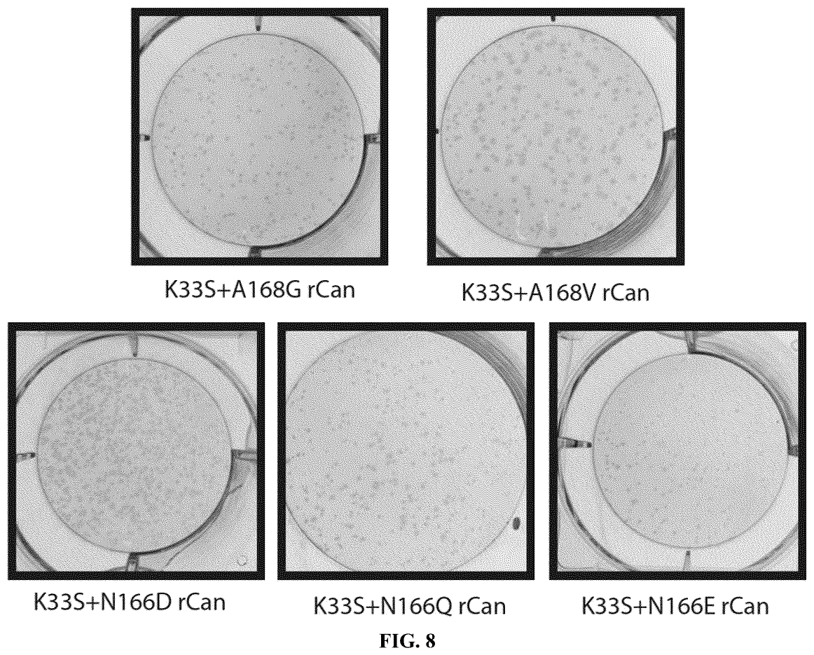

Serial dilutions of stocks prepared from rescued rCan bearing K33S mutation and a mutation at positions 166 or 168 were used to infect Vero cell monolayers in 6-well cluster dishes. Cultures were then overlayed with agarose and incubated for 5 days, at which time wells were fixed in cold 2% paraformaldehyde and the agarose removed. Monolayers were stained for infection using an anti-nucleoprotein (NP) monoclonal antibody (AG12; Sanchez et al 1989 J Gen Virol 70:1125-32), a secondary a horseradish-peroxidase conjugated anti-mouse antibody and DAB substrate. Images demonstrate the robust viability of these viruses (FIG. 8).

K33S+N166D rCan and K33S+N166Q rCan are somewhat less robust than K33S rCan, whereas K33S+166E is more debilitated. K33S+N166L rCan could not be rescued, suggesting a significant growth defect.

What is claimed:

1. An attenuated Junín virus (JUNV) comprising:
at least one mutation altering a NxT/S glycosylation motif in a region spanning residues 150-175 of the GPC protein thereby inactivating a glycosylation site; and
a mutation protecting an attenuating mutation in the GPC protein's GP2 subunit.

2. The attenuated JUNV of claim 1, wherein the attenuated JUNV comprises at least three mutations in its GPC protein, wherein:
a first mutation alters the NxT/S glycosylation motif in a region spanning residues 150-175 of the GPC protein;
a second mutation affects GPC protein's stable signal peptide subunit; and
a third mutation targets the attenuating locus in the GPC protein's GP2 subunit, wherein the third mutation replaces a phenylalanine residue with an isoleucine.

3. The attenuated JUNV of claim 1, wherein the attenuated JUNV comprises at least three mutations in its GPC protein, wherein:
a first mutation and a second mutation alter the NxT/S glycosylation motif in a region spanning residues 150-175 of the GPC protein; and
a third mutation targeting the attenuating locus in the GPC protein's GP2 subunit, wherein the third mutation replaces a phenylalanine residue with an isoleucine.

4. The attenuated JUNV of claim 3, wherein the first mutation replaces the T/S residue of the NxT/S glycosylation motif with a glycine or valine.

5. The attenuated JUNV of claim 3, wherein the mutations are described in accordance with the amino acid sequence of clinical isolate XJ,
the first mutation is T168G or T168V;
the second mutation is N166Q, N166D, or N166E; and
the third mutation is F427I.

6. The attenuated JUNV of claim 5, further comprising a fourth mutation selected from the group consisting of: K33S, K33A, K33Q, and K33E.

7. The attenuated JUNV of claim 6, wherein the GPC protein has an amino acid sequence set forth in any one of SEQ ID NO:1 or SEQ NOs: 15-18.

8. The attenuated JUNV of claim 2, wherein:
the first mutation alters the N residue of the NxT/S glycosylation motif to an amino acid selected from the group consisting of: glutamine, aspartic acid, and glutamic acid; and
the second mutation replaces a lysine residue in the stable signal peptide subunit.

9. The attenuated JUNV of claim 8, wherein the mutations are described in accordance with the amino acid sequence of clinical isolate XJ,
the first mutation is N166Q, N166D, or N166E;
the second mutation is K33S, K33A, K33Q, and K33E; and
the third mutation is F427I.

10. The attenuated JUNV of claim 9, wherein the GPC protein has an amino acid sequence set forth in any one of SEQ ID NO:1, SEQ ID NO:2, SEQ NOs: 9-12, or SEQ NOs: 15-18.

11. The attenuated JUNV of claim 2, wherein:
the first mutation replaces the T/S residue of the NxT/S glycosylation motif with a glycine or valine; and
the second mutation replaces a lysine residue in the stable signal peptide subunit.

12. The attenuated JUNV of claim 11, wherein the GPC protein has an amino acid sequence set forth in SEQ ID NO:1, or SEQ NOs: 13-18.

13. A vaccine composition comprising the attenuated JUNV of claim 1 and a pharmaceutically acceptable additive.

14. A composition for diagnosing a subject has been infected with JUNV, the composition comprising:
the attenuated JUNV of claim 1 or protein isolated therefrom; and
a surface, wherein the reversion-resistant JUNV is affixed to the surface.

15. A method of limiting reversion to virulence of Candid#1 vaccine strain, the method comprising:
editing the genome of the Candid#1 vaccine strain resulting in inactivation of a NxT/S glycosylation motif in a region spanning residues 150-175 of the Junín virus GPC protein to introduce at least one amino acid substitution selected from the group consisting of: N>Q, N>D, N>E, A>G, and A>V;
editing the genome of the Candid#1 vaccine strain resulting in a mutation at a position corresponding to residue 33 of the Junín virus GPC protein; and
expressing the edited genome of the Candid#1 vaccine strain in a mammalian cell thereby resulting in replication of an attenuated JUNV, wherein the attenuated JUNV expresses a GPC protein with mutations at residues 33 and 427 and lacks NxT/S glycosylation motif in a region spanning residues 150-175.

16. The method of claim 15, wherein the step of editing the genome editing the genome of the JUNV resulting in removal of the NxT/S glycosylation motif in a region spanning residues 150-175 of the Junín virus GPC protein introduces an amino acid substitution in the GPC protein selected from the group consisting of: A168G and A168V.

17. The method of claim 15, wherein the step of editing the genome editing the genome of the JUNV resulting in removal of a NxT/S glycosylation motif in a region spanning residues 150-175 of the Junín virus GPC protein introduces an amino acid substitution in the GPC protein selected from the group consisting of: N166Q, N166D, and N166E.

18. The method of claim 15, wherein the step of editing the genome editing the genome of the JUNV resulting in removal of a NxT/S glycosylation motif in a region spanning residues 150-175 of the Junín virus GPC protein introduces a first amino acid substitution in the GPC protein selected from the group consisting of: N166Q, N166D, and N166E, and a second amino acid substitution in the GPC protein selected from the group consisting of: A168G and A168V.

19. The method of claim 16, wherein the step of editing the genome of the JUNV resulting in a mutation at a position corresponding to residue 33 replaces the lysine of residue 33 with serine, alanine, glutamine, or glutamic acid.

20. The method of claim 17, wherein the step of editing the genome of the JUNV resulting in a mutation at a position corresponding to residue 33 replaces the lysine of residue 33 with serine, alanine, glutamine, or glutamic acid.


SEQUENCE LISTING (18 sequences)

SEQ ID NO: 1 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRQRGKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 2 - protein, 485 aa, Mammarenavirus juninense
MGQFISFMQEIPTFLQEALNIALVAVSLIAIIKGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRQRGKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 3 - DNA, 64 bp, synthetic construct
  source 1..64: mol_type = other DNA
gcatctagacccaccatttctgtgtagggagcgtgcaaagacagaaggcttcatctttcaagtc

SEQ ID NO: 4 - DNA, 63 bp, synthetic construct
  source 1..63: mol_type = other DNA
gcatctagacccaccatttctgtgtagggaccgtgcaaagacagaaggcttcatctttcaagt

SEQ ID NO: 5 - DNA, 64 bp, synthetic construct
  source 1..64: mol_type = other DNA
gcatctagacccaccatttctgtgtaggctgcgtgcaaagacagaaggcttcatctttcaagtc

SEQ ID NO: 6 - DNA, 64 bp, synthetic construct
  source 1..64: mol_type = other DNA
gcatctagacccaccatttctgtgtaggcaacgtgcaaagacagaaggcttcatctttcaagtc

SEQ ID NO: 7 - DNA, 64 bp, synthetic construct
  source 1..64: mol_type = other DNA
gcatctagacccaccatttctgtgtaggaaccgtgtaaagacagaaggcttcatctttcaagtc

SEQ ID NO: 8 - DNA, 64 bp, synthetic construct
  source 1..64: mol_type = other DNA
gcatctagacccaccatttctgtgtaggaaccgtggaaagacagaaggcttcatctttcaagtc

SEQ ID NO: 9 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRDRAKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 10 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRQRAKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 11 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRERAKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 12 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRLRAKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 13 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRNRVKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 14 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRNRGKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 15 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRDRGKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 16 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRDRVKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH

SEQ ID NO: 17 - protein, 485 aa, Mammarenavirus juninense; identical to SEQ ID NO: 1

SEQ ID NO: 18 - protein, 485 aa, Mammarenavirus juninense
MGQFISFMQEIPTFLQEALNIALVAVSLIAIISGIVNLYKSGLFQFFVFLALAGRSCTEEAFKIGLHTEFQTVSFSMVGLFSNNPHDLPLLCTLNKSHLYIKGGNASFQISFDDIAVLLPQYDVIIQHPADMSWCSKSDDQIWLSQWFMNAVGHDWHLDPPFLCRQRVKTEGFIFQVNTSKTGVNGNYAKKFKTGMHHLYREYPDPCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICIWSTVFFTASLFLHLVGIPSHRHIRGEACPLPHRLNSLGGCRCGKYPNLKKPTVWRRGH